SEQUENCE LISTING (5 sequences)

SEQ ID NO: 1 - protein, 16 aa, Homo sapiens
DALSSVQESQVAQQAR

SEQ ID NO: 2 - protein, 19 aa, Homo sapiens
FSEFWDLDPEVRPTSAVAA

SEQ ID NO: 3 - protein, 11 aa, Homo sapiens
GWVTDGFSSLK

SEQ ID NO: 4 - protein, 21 aa, Homo sapiens
DKFSEFWDLDPEVRPTSAVAA

SEQ ID NO: 5 - protein, 99 aa, Homo sapiens
  SIGNAL (1)..(20)
MQPRVLLVVALLALLASARASEAEDASLLSFMQGYMKHATKTAKDALSSVQESQVAQQARGWVTDGFSSLKDYWSTVKDKFSEFWDLDPEVRPASAVAA

(19) United States
(12) Patent Application Publication
(10) Pub. No.: US 2014/0051079 A1
(43) Pub. Date: Feb. 20, 2014

(54) METHOD FOR PREDICTING THE RESPONSE TO A TREATMENT AGAINST HEPATITIS C

(71) Applicants: Sylvain Lehmann, Castelnau-le-Lez (FR); Stephane Roche, Montpellier (FR); Jacques Ducos, Montpellier (FR); Laurent Tiers, Montpellier (FR)

(72) Inventors: Sylvain Lehmann, Castelnau-le-Lez (FR); Stephane Roche, Montpellier (FR); Jacques Ducos, Montpellier (FR); Laurent Tiers, Montpellier (FR)

(73) Assignee: CENTRE HOSPITALIER UNIVERSITAIRE DE MONTPELLIER, Montpellier (FR)

(21) Appl. No.: 13/993,991

(22) PCT Filed: Dec. 14, 2011
(86) PCT No.: PCT/FR11/52989

§ 371 Date: Oct. 31, 2013

(30) Foreign Application Priority Data
Dec. 15, 2010 (FR) 1060568

(51) Int. Cl. G01N 33/92; G01N 33/576; C12Q 1/68

(52) U.S. Cl. CPC G01N 33/92; C12Q 1/6883; G01N 33/5767
USPC 435/6.11; 436/501

(57) ABSTRACT

The invention relates to a method for predicting the response to an interferon-based treatment in a patient infected with hepatitis C virus. This method consists in determining the presence of apolipoprotein CIII and/or of a multimeric form of human serum albumin in a sample of biological fluid from the patient.

### TECHNICAL FIELD OF THE INVENTION

The present invention relates to the field of diagnosis. More particularly, the invention relates to a method and a kit for predicting the efficacy of a hepatitis C treatment.

### PRIOR ART

According to the Bulletin épidémiologique hebdomadaire [Weekly epidemiological bulletin] of the Institut de Veille Sanitaire [French Institute for Public Health Surveillance] dated Jul. 1, 2008, the annual number of deaths associated with hepatitis C virus (HCV) in 2001 was 3618 (respectively 6.1 deaths per 100 000 inhabitants). The annual number of deaths attributable to HCV was 2646 (4.5 per 100 000 inhabitants). 95% had cirrhosis and 33% a hepatocellular carcinoma. This mortality should increase in years to come given that the very large increase in the number of instances of primary liver cancer in Western countries is to a large extent linked to hepatitis C virus.

Currently, three types of therapies are available for treating HCV infection:
treatment with interferon, which is effective in approximately 20% of individuals infected with HCV;
the pegylated interferon-ribavirin combination, which is effective in approximately 50% of individuals infected with HCV;
interferon developed via biological techniques, which is effective in approximately 60 to 70% of individuals infected with HCV.

All these treatments, which are administered by injection, have adverse effects such as fever, headaches, fatigue, nausea and vomiting. In addition, the treatment modifies the blood count.

In patients suffering from chronic hepatitis C, the pegylated interferon-ribavirin combination leads to a prolonged viral response in 55% of cases. Obtaining a prolonged virological response is the principle objective of the treatment for chronic hepatitis C (approximately 60 to 80% of patients infected with HCV). It is associated with the disappearance of the histological activity and possibly of hepatic fibrosis, and also with a reduction in the risk of hepatocellular carcinoma.

Non-responder patients (45%) can be identified by detection of the viral load at best at the week-12 tests, or even at the arrest of treatment (6 or 12 months according to genotype). The viral load present at week 12 can condition the arrest of therapy.

Non-response to the treatment is a problem in particular for patients infected with genotype 1 of the virus, who respond favorably only in 40-50% of cases, compared with patients infected with genotypes 2 and 3, who have 80-90% response to the treatment.

A recent study has demonstrated factors which influence the response to the treatment, including age, viral genotype, and body mass index (Elefsiniotis et al., 2008). However, these elements are not effective enough to dictate a therapeutic choice.

When studying, in vitro, the mechanisms of resistance to HCV treatment, it appears that various viral proteins (E2, NS3/4A and NS5A) may play a role therein (Hofmann et al., 2005). In the majority of clinical studies, it has not however been possible to link the variations in sequence of these proteins to the response to the treatment (Wohnsland et al., 2007). In parallel, overall genomic approaches (chip, SNP—single nucleotide polymorphism) have been carried out in order to find predictive markers for response to the treatment. Certain polymorphisms of the IL10 promoter (Morgan et al., 2008) have been suggested, but it is in fact a marker known as IL28B (Ge et al., 2009) which appears to currently show an advantage clinically.

There is therefore a need to be able to provide a reliable predictive marker for response to the interferon-based treatment, which is a restrictive and expensive treatment. Such a marker, termed “theranostic”, would be very useful and would make it possible to add an important element to the therapeutic choice offered to the patients infected with hepatitis C virus.

### DESCRIPTION OF THE FIGURES

FIGS. 1 and 2 represent the distribution of the level of apo-C3 and, respectively, of the level of HSA2, in patients who are responders and non-responders to an interferon-based treatment.

FIG. 3 represents the area under the curve of HSA2 and/or of apo-C3 (for the responder patients).

### DESCRIPTION OF THE INVENTION

The inventors have discovered, and this is the basis of the invention, that there is a correlation between the presence of apolipoprotein CIII (hereinafter, apo-C3) and/or of human serum albumin dimer (hereinafter, HSA2) and the response or non-response to an interferon-based therapy in patients infected with hepatitis C virus.

Thus, according to a first aspect, the invention relates to an in vitro method for predicting the response to an interferon-based treatment in a patient infected with HCV, said method comprising a step of measuring the level of apo-C3 and/or of HSA2 in a biological sample from said patient prior to any treatment. Typically, the biological sample is a serum or plasma sample.

The response to the interferon-based treatment can take various forms: (i) an improvement in the clinical situation during and after the treatment, (ii) an improvement in the clinical situation during the treatment, but a relapse after the treatment, or (iii) no improvement in the clinical situation during or after the treatment. The patients targeted in (i) will subsequently be described as “responder” (R) patients, whereas the patients targeted in (ii) and (iii) will subsequently be described as “non-responder” (NR) patients. The improvement in the clinical situation of a patient is reflected by a (statistically) significant decrease in the viral load of said patient, which is measured by techniques known per se.

For the purposes of the present invention, the term “interferon” is intended to mean interferon-alpha (alpha 2a or 2b) and its pegylated or non-pegylated forms, interferon-beta (beta 1a or 1b) and also interferon-gamma. According to one embodiment, the interferon-based treatment is a treatment based on pegylated or non-pegylated interferon-alpha, optionally in combination with ribavirin. According to another embodiment, the interferon-based treatment is a treatment based on interferon-beta.

According to one embodiment, the method in accordance with the invention also comprises comparing the measured level of apo-C3 with a threshold level of apo-C3, a measured level of apo-C3 which is less than or equal to the threshold level being predictive of a response to the interferon-based treatment.

apo-C3 is an 8.8 kDa polypeptide composed of a mature sequence of 79 amino acids, preceded by a signal peptide of 20 amino acids. The enzymatic digestion by thrombin during coagulation which is triggered during the preparation of serum (but not of plasma) cleaves the mature sequence so as to generate a fragment, corresponding to amino acids 41-79 (COOH terminal) (Catapano et al. 1987). This fragment is detected in serum samples, and the assaying thereof therefore corresponds in this case to the blood apo-C3 level.

The threshold level of apo-C3 is established from a population of patients infected with HCV, from whom a biological sample has been taken and analyzed before the beginning of the interferon-based treatment and who have subsequently been categorized as responder or non-responder patients, typically by detection of their viral load at the week-12 tests, as indicated above. The threshold level is the value which makes it possible to have the best statistical distribution of the population studied between the R group and the NR group using a given technology; it is subsequently possible, in order to determine whether any new patient belongs to the R or NR group, to measure said patient's level of apo-C3 using the same technology and to compare this level to the threshold level. Of course, the threshold level of apo-C3 is capable of varying according to the technique and the equipment used for measuring the apo-C3 levels, and those skilled in the art will be able to determine this threshold level according to the equipment at their disposal.

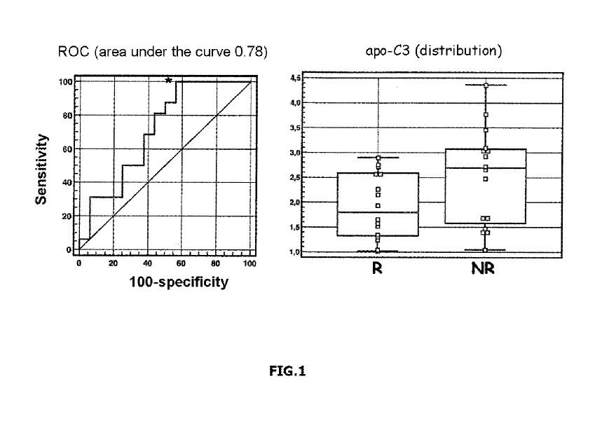

As can be noted on reading FIG. 1, there is a statistically significant threshold of apo-C3 below which it is possible to predict with good certainty (high specificity) that the patients infected with HCV will respond to an interferon-based treatment.

According to another embodiment, which can be combined with the previous embodiment, the method in accordance with the invention also comprises comparing the measured level of HSA2 with a threshold level of HSA2, a measured level of HSA2 which is equal to or greater than the threshold level being predictive of a response to the interferon-based treatment.

The threshold level of HSA2 is established from a population of patients infected with HCV, from whom a biological sample has been taken and analyzed before the beginning of the interferon-based treatment and who have then been categorized as responder or non-responder patients, typically by detection of their viral load at the week-12 tests, as indicated above. The threshold level is the value which makes it possible to obtain the best statistical distribution of the population studied between the R group and the NR group using a given technology; it is then possible, in order to determine whether any new patient belongs to the R or NR group, to measure the HSA2 level of said patient using the same technology and to compare this level to the threshold level. Of course, the threshold level of HSA2 is capable of varying according to the technique and equipment used to measure the HSA2 levels, and those skilled in the art will be able to determine this threshold level according to the equipment available to them.

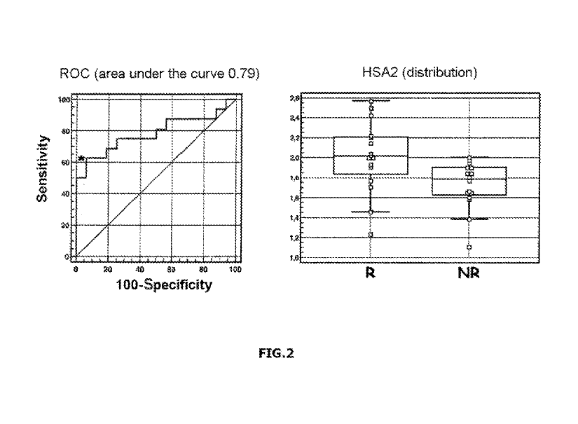

As can be noted on reading FIG. 2, there is a statistically significant HSA2 threshold above which it is possible to predict with an excellent specificity (93.8%) that the patients infected with HCV will respond to an interferon-based treatment.

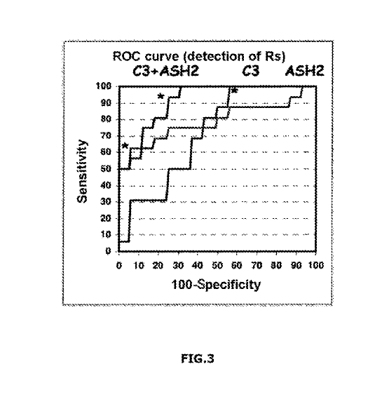

It is possible to improve the sensitivity of the method of the invention by jointly measuring the HSA2 and apo-C3 levels. As can be noted on reading FIG. 3, such a joint measurement makes it possible to detect 93.8% of responders with a good specificity (75%).

The apo-C3 and HSA2 levels can be measured using a mass spectrometry technique called SELDI-TOF (surface enhanced laser desorption/ionization time-of-flight mass spectrometry). This technique makes it possible to retain proteins on various chromatographic surfaces and to detect them by mass spectrometry. It is thus possible to rapidly obtain a protein expression profile which will be analyzed by virtue of bioinformatic means. The interesting peaks are selected and the corresponding proteins identified after biochemical purification according to techniques well known to those skilled in the art. It is then possible to quantify the level of the proteins identified according to techniques also well known to those skilled in the art (such as quantitative mass spectrometry or immunodetection).

According to another embodiment, the method in accordance with the invention also comprises measuring the expression or the expression level of at least one of the following markers:
the IL28B genetic marker, in particular the rs12979860 CC polymorphism and/or the rs8099917 TT polymorphism of said marker;
the genes listed in table 4 of patent application US 2005/028279, in particular those chosen from the group comprising ADAR, IFI27, IFI44, OAS3, MX1, MX2, PRKR, IFIT4, TRIM22 and G1P2;
HCV-1 or HCV-4 (US 2010/0158866);
the polynucleotides of sequence No. 1, No. 2, No. 3 or No. 4 of application WO 01/71007;
the IP-10 protein (WO 2008/032210), also known as CXCL10;
the genes chosen from the group comprising KIR3DL3, KIR3DL2, KIR3DL1, KIR2DL1, KIR2DL2, KIR2DL3, KLRG1, KIR3DS1, CD160, HLA-A, HLA-B, HLA-C, HLA-F, HLA-G, IFI27, TNFRSF17, IFI6, OAS2, ISG15, OAS3 and IFIT1 (patent application WO 2010/076788).

Advantageously, the method in accordance with the invention comprises, in addition to measuring the level of apo-C3 and/or of HSA2, detecting the polymorphisms of IL28B and/or measuring the expression level of CXCL10.

The method in accordance with the invention therefore makes it possible to predict the response to an interferon-based treatment in a patient infected with HCV using a biological sample, typically a serum or plasma sample, taken from the patient before the institution of the treatment. It is therefore understood that the diagnosis of infection with HCV has been made, by means of conventional tests (for example by measuring the viral load, determined by means of the RNA levels, and/or the level of capsid antigen), and that the purpose of the method in accordance with the invention is to determine whether an interferon-based treatment is appropriate, i.e. whether or not a patient is liable to obtain a benefit from such a treatment.

By virtue of the method in accordance with the invention, it is possible to propose to a patient infected with HCV, and in particular with genotype 1 of the virus, an alternative treatment to the interferon-based treatment as soon as it has been determined (by the simple and reliable means previously described) that said patient will not respond to said interferon-based treatment. Such an alternative treatment may comprise administration of at least one active ingredient that is of use in the treatment of HCV, chosen in particular from protease inhibitors, polymerase inhibitors and NS5A inhibitors.

Moreover, the fact that a patient is or is not liable to respond to an interferon-based treatment will condition, in the medium and long term, the progression of said patient's disease, which will enable, where appropriate, a better organization of the management thereof by the competent medical teams.

According to a second aspect, the invention relates to a kit for carrying out the abovementioned method. This kit comprises:
standards for establishing a reference curve; and/or
one or more specific reagents (antibodies, buffers, visualizing reagents) required for carrying out the quantitative assaying of apo-C3 and/or of HSA2; and optionally
instructions for carrying out the quantitative assaying of apo-C3 and/or of HSA2.

In one embodiment, the kit according to the invention also comprises a specific reagent required for the assaying of CXCL10.

The invention is illustrated by the following examples, given by way of indication.

### Example 1

### Demonstration of the Presence of Human Serum Albumin Dimer and of Apolipoprotein CIII in Patients Infected with HCV

In the context of a clinical research protocol of the Montpellier Centre Hospitalier Regional Universitaire [Regional University Hospital Center] which has been ethically validated (authorization of the Comité de Protection des Personnes pour la Recherche Biomédicale—CPPRB [Ethics Committee relating to Biomedical Research]), samples were taken from patients infected with HCV (16 of genotype 1, 3 of genotype 2, 8 of genotype 3 and 1 of genotype 4, the other genotypes being unknown) prior to institution of an interferon-based treatment. The samples were analyzed by SELDI mass spectrometry.

In order to demonstrate the peaks of interest, the various samples were deposited on a CM10 array and Q10 array, pH 7. The protocol was the following:

a) sample preparation: 10 μl of serum were placed in 15 μl of a urea CHAPS solution and stirring was carried out for 15 min in order to denature the proteins. 7.5 μl of the mixture were diluted in 300 μl of buffer (100 mM Tris, pH 7+0.1% Triton);
b) array incubation: the 2 arrays (CM10 and Q10) were placed in a bioprocessor. 150 μl of buffer (Tris, pH 7+0.1% Triton) were added to the spots. Incubation was carried out for 5 min with shaking (500 rpm) and then the buffer was removed and the bioprocessor was wiped on absorbent paper;
c) sample deposit: the diluted samples were deposited on the spots (8 samples per array);
d) washing: the spots were washed twice for 5 min with 150 μl of Tris, pH 7+0.1% Triton buffer in order to remove the proteins not bound to the biochemical surface, and then once for 5 min with 150 μl of Tris buffer, pH 7, without Triton, in order to remove the detergent. The arrays were then rapidly rinsed in 10 ml of 5 mM HEPES, pH 7, in order to remove all the salts, and were left to dry for 10 min;
e) analysis: 2×0.8 μl of SPA matrix were added (a period of 10 min was left between the 2 applications in order to allow drying). The analysis by SELDI-TOF was carried out using the Protein Chip SELDI System PCS 4000 mass spectrometer from the company Biorad. The arrays were read at low molecular weight (3500-20 000 Da) and at high molecular weight (20 000-150 000 Da) according to two different reading protocols:
low molecular weight (LMW): focusing on 10 KDa; laser energy 2100 nJ and over a weight range of 0-200 000 Da, with a warning shot at 2300 and ¼ of the spot is read.
high molecular weight (HMW): focusing on 130 kDa, laser energy 2300 nJ and over a weight range of 0-200 000 Da, with a warning shot at 2500 and ¼ of the spot is read.

After having passed all the samples through SELDI-TOF, a spectrum was obtained for each sample.

The analysis was carried out principally by means of the Protein chip Data manager software. Before analyzing the spectra, it is necessary beforehand:
to align the various spectra;
to calculate the average background noise in a selected weight range;
to adjust the baseline as well as possible, before subtracting it from the spectrum;
to standardize the intensities of the spectra with respect to total ion current (TIC).

A first series of analysis on 32 samples was carried out, making it possible to select several SELDI peaks of interest (by statistical difference between R and NR groups based on a Student's test and a Mann-Whitney test with a minimum of p<0.05). A validation using 16 new samples made it possible to confirm the interest of certain peaks (using the Student's test and Mann-Whitney test and by calculating the sensitivity and specificity obtained by taking two peaks under consideration in order to categorize the population) and, in the end, to select 5 of them.

Among these 5 peaks, one of them, called p10, was identified by mass spectrometry after biochemical purification. A second of these 5 peaks, called p4, was also identified by mass spectrometry after biochemical purification.

The purification of the p10 and p4 markers could be carried out by reproducing, on larger serum volumes, in spin columns, the SELDI capture conditions. The fractions obtained were analyzed by SELDI in order to verify the presence of the marker of interest, and they were then loaded onto an electrophoresis gel. After staining of the gel, the zone where the marker was expected owing to its molecular weight (MW) was excised. A final verification of the presence of the marker eluted from the gel was carried out before its sequence was obtained by LC-MS/MS analysis.

With regard to p10, human serum albumin (HSA) could be identified. In the knowledge that albumin has an MW of 66 kDa and that p10 has an MW of 132 kDa, it can be concluded that p10 corresponds to a dimer of HSA. As for p4, it corresponds to a truncated form of apolipoprotein CIII. For the latter marker, the peptides sequenced were the following:

(SEQ ID No. 1)
DALSSVQESQVAQQAR
(SEQ ID No. 2)
FSEFWDLDPEVRPTSAVAA
(SEQ ID No. 3)
GWVTDGFSSLK
(SEQ ID No. 4)
DKFSEFWDLDPEVRPTSAVAA.

The alignment of these peptides on the sequence of apo-C3, preceded by its signal peptide, is shown below:

(SEQ ID No. 5)
MQPRVLLVVA LLALLASARA SEAEDASLLS FMQGYMKHAT
KTAKDALSSV QESQVAQQAR GWVTDGFSSL KDYWSTVKDK
FSEFWDLDPE VRPASAVAA.

The sequenced part therefore corresponds to fragment 41-79 of apo-C3 which is found in serum samples subsequent to cleavage by thrombin during coagulation (Catapano et al., 1987).

### Example 2

### Correlation Between Presence of the Markers of Interest and Response or Non-Response to the Interferon-Based Treatment

The patients mentioned in example 1 were treated with pegylated interferon-alpha+ribavirin. A patient follow-up was carried out 12 weeks after the beginning of the treatment, at which time new samples were taken in order to determine
firstly, the responder (R) patients and the non-responder (NR) patients; and
secondly, for each category of patients, whether there is a (statistically significant) correlation between the levels of HSA2 and/or of apo-C3 determined before the institution of the treatment and the response or non-response to said treatment.

As can be noted on reading table 1 and FIG. 1, there is a statistically significant difference in the level of apo-C3 (determined before the institution of the treatment) between responders and non-responders (p<0.05 in the t-test and p<0.05 in the Kruskal-Wallis test). An apo-C3 level below the threshold, in arbitrary units, of 2.9 makes it possible to detect 100% of responders with a specificity of 43.8% (see table 2), i.e. no non-responder is detected beyond this threshold.

Likewise, it can be noted on reading table 1 and FIG. 1 that there is a statistically significant difference in the level of HSA2 (determined before institution of the treatment) between responders and non-responders (p<0.01 in the t-test and p<0.006 in the Kruskal-Wallis test). An HSA2 level above the threshold of detection, in arbitrary units, of 1.9 makes it possible to detect 62.5% of responders with an excellent specificity of 93.8% (see table 2), i.e. with very few false positives beyond this threshold.

Finally, a combination of these two markers, taking as criterion the thresholds for these two parameters, makes it possible to further improve the detection results with an ROC area of 0.91 (see table 2 and FIG. 3).

TABLE 1
apo-C3 and HSA2 values measured for the R and NR patients

| Group | apo-C3 | HSA2 | Group | apo-C3 | HSA2 |
|---|---|---|---|---|---|
| R | 1.35 | 1.46 | NR | 2.66 | 1.81 |
| R | 2.57 | 2.15 | NR | 2.72 | 1.67 |
| R | 2.75 | 2.49 | NR | 3.46 | 1.84 |
| R | 2.15 | 2.00 | NR | 3.04 | 1.95 |
| R | 2.90 | 1.71 | NR | 3.77 | 1.90 |
| R | 2.59 | 2.42 | NR | 3.10 | 1.98 |
| R | 1.94 | 2.03 | NR | 3.05 | 2.00 |
| R | 1.65 | 1.77 | NR | 4.36 | 1.61 |
| R | 2.26 | 1.91 | NR | 1.47 | 1.11 |
| R | 1.01 | 1.93 | NR | 1.40 | 1.38 |
| R | 1.52 | 2.21 | NR | 1.69 | 1.76 |
| R | 2.71 | 2.57 | NR | 2.48 | 1.65 |
| R | 1.24 | 1.23 | NR | 2.92 | 1.59 |
| R | 1.30 | 2.20 | NR | 1.41 | 1.85 |
| R | 1.05 | 2.00 | NR | 1.04 | 1.65 |
| R | 1.57 | 2.04 | NR | 1.68 | 1.91 |

TABLE 2

| Peak | Sensitivity | Specificity | ROC |
|---|---|---|---|
| apo-C3 | 100 | 43.8 | 0.72 |
| HSA2 | 62.5 | 93.8 | 0.79 |
| apo-C3 + HSA2 | 93.8 | 75 | 0.91 |

### LITERATURE REFERENCES

Catapano A L. (1987) Activation of lipoprotein lipase by apolipoprotein C-II is modulated by the COOH terminal region of apolipoprotein C-III. Chem Phys Lipids. 1987 October; 45(1): 39-47
Elefsiniotis, I. S., Pavlidis, C., Ketikoglou, I., Koutsounas, S., Scarmeas, N., Pantazis, K. D., Moulakakis, A. & Tsianos, E. V. (2008). Patient's age modifies the impact of the proposed predictors of sustained virological response in chronic hepatitis C patients treated with PEG-interferon plus ribavirin. Eur J Intern Med 19, 266-270.
Hofmann, W. P., Dries, V., Herrmann, E., Gartner, B., Zeuzem, S. & Sarrazin, C. (2005). Comparison of transcription mediated amplification (TMA) and reverse transcription polymerase chain reaction (RT-PCR) for detection of hepatitis C virus RNA in liver tissue. J Clin Virol 32, 289-293.
Ge D, Fellay J, Thompson A J, Simon J S, Shianna K V, Urban T J, et al. (2009) Genetic variation in IL28B predicts hepatitis C treatment-induced viral clearance. Nature 2009; 461: 399-401.
Morgan, T. R., Lambrecht, R. W., Bonkovsky, H. L., Chung, R. T., Naishadham, D., Sterling, R. K., Fontana, R. J., Lee, W. M., Ghany, M. G., Wright, E. C. & O'Brien, T. R. (2008). DNA polymorphisms and response to treatment in patients with chronic hepatitis C: results from the HALT-C trial. J Hepatol 49, 548-556.
Wohnsland, A., Hofmann, W. P. & Sarrazin, C. (2007). Viral determinants of resistance to treatment in patients with hepatitis C. Clin Microbiol Rev 20, 23-38.

1-12. (canceled)

13. An in vitro method for predicting the response to an interferon-based treatment in a patient infected with hepatitis C virus (HCV), wherein said method comprises measuring the level of apo-C3 in a biological sample from said patient prior to any treatment.

14. The method of claim 13, which further comprises comparing the measured level of apo-C3 with a threshold level of apo-C3, wherein a measured level of apo-C3 which is less than or equal to the threshold level is predictive of a response to the interferon-based treatment.

15. The method of claim 13, wherein the patient is infected with HCV genotype 1.

16. The method of claim 13, wherein the interferon-based treatment comprises a treatment with an interferon selected from the group consisting of pegylated or non-pegylated interferon-alpha, interferon-beta and interferon-gamma.

17. The method of claim 16, wherein the interferon-based treatment comprises a treatment with pegylated or non-pegylated interferon-alpha.

18. The method of claim 16, wherein the interferon-based treatment comprises a treatment with pegylated or non-pegylated interferon-alpha in combination with ribavirin.

19. The method of claim 13, wherein the biological sample is a serum or plasma sample.

20. The method of claim 13, which further comprises a step of detecting IL28B polymorphisms.

21. The method of claim 1, which further comprises a step of measuring the expression level of CXCL10.

22. An in vitro method for predicting the response to an interferon-based treatment in a patient infected with hepatitis C virus (HCV), wherein said method comprises measuring the level of apo-C3 and measuring the level of HSA2 in a biological sample from said patient prior to any treatment.

23. The method of claim 22, which further comprises comparing the measured level of apo-C3 with a threshold level of apo-C3, wherein a measured level of apo-C3 which is less than or equal to the threshold level is predictive of a response to the interferon-based treatment.

24. The method of claim 22, which further comprises comparing the measured level of HSA2 with a threshold level of HSA2, wherein a measured level of HSA2 which is equal to or greater than the threshold level is predictive of a response to the interferon-based treatment.

25. The method of claim 22, wherein the patient is infected with HCV genotype 1.

26. The method of claim 22, wherein the interferon-based treatment comprises a treatment with an interferon selected from the group consisting of pegylated or non-pegylated interferon-alpha, interferon-beta and interferon-gamma.

27. The method of claim 26, wherein the interferon-based treatment comprises a treatment with pegylated or non-pegylated interferon-alpha.

28. The method of claim 26, wherein the interferon-based treatment comprises a treatment with pegylated or non-pegylated interferon-alpha in combination with ribavirin.

29. The method of claim 22, wherein the biological sample is a serum or plasma sample.

30. The method of claim 22, which further comprises a step of detecting IL28B polymorphisms.

31. The method of claim 22, which further comprises a step of measuring the expression level of CXCL10.

32. An in vitro method for predicting the response to an interferon-based treatment in a patient infected with hepatitis C virus (HCV), wherein said method comprises measuring the level of HSA2 in a biological sample from said patient prior to any treatment.

33. The method of claim 32, which further comprises comparing the measured level of HSA2 with a threshold level of HSA2, wherein a measured level of HSA2 which is equal to or greater than the threshold level is predictive of a response to the interferon-based treatment.

34. The method of claim 32, wherein the patient is infected with HCV genotype 1.

35. The method of claim 32, wherein the interferon-based treatment comprises a treatment with an interferon selected from the group consisting of pegylated or non-pegylated interferon-alpha, interferon-beta and interferon-gamma.

36. The method of claim 32, wherein the interferon-based treatment comprises a treatment with pegylated or non-pegylated interferon-alpha.

37. The method of claim 32, wherein the interferon-based treatment comprises a treatment with pegylated or non-pegylated interferon-alpha in combination with ribavirin.

38. The method of claim 32, wherein the biological sample is a serum or plasma sample.

39. The method of claim 32, which further comprises a step of detecting IL28B polymorphisms.

40. The method of claim 32, which further comprises a step of measuring the expression level of CXCL10.

41. A kit comprising:
at least one reagent selected from the group consisting of specific reagents required for carrying out the quantitative assay of apo-C3 and specific reagents required for carrying out the quantitative assay of HSA2;
standards for establishing a reference curve.

42. The kit of claim 41, which further comprises one or more specific reagents required for carrying out the quantitative assay of CXCL10.
